(19) United States
(12) Plant Patent Application Publication
(10) Pub. No.: US 2009/0055979 P1
(43) Pub. Date: Feb. 26, 2009

(54) Tristaniopsis laurina plant named 'DOW10'

(76) Inventor: Todd Anthony Layt, Richmond (AU)

Correspondence Address: Sam R. McCoy Jr., P.O. Box 2108, Mount Pleasant, SC, 29465, US

(21) Appl. No.: 11/891,490

(22) Filed: Aug. 10, 2007

Related U.S. Application Data
(60) Provisional application No. 60/836,877, filed on Aug. 10, 2006.

(51) Int. Cl. A01H 5/00

(52) U.S. Cl. PLT/216

(57) ABSTRACT

‘DOW10’ is a distinctive variety of Tristaniopsis laurina, which is characterized by its combination of light brown bark on mature stems shedding to reveal light gray brown colored immature bark and large, glossy, dark green leaves combined with a vigorous growth rate.

### LATIN NAME OF THE GENUS AND SPECIES

The Latin name of the novel variety disclosed herein is Tristaniopsis laurina ‘DOW10’.

### VARIETY DENOMINATION

The inventive variety of Tristaniopsis laurina disclosed herein has been given the variety denomination ‘DOW10’.

### BACKGROUND OF THE INVENTION

The present invention relates to a new and distinct variety of evergreen perennial Tristaniopsis laurina, which has been named ‘DOW10’. Tristaniopsis laurina is an Australian native tree that grows to a height usually up to 10 metres tall and is used as an ornamental tree in landscape and amenity horticulture. Tristaniopsis laurina has brown bark that sheds in strips to reveal smooth light gray brown colored immature bark underneath. Large glossy green leaves typically become crowded towards the end of branches. Flowers are yellow, appearing in early summer and are followed by capsules that produce winged seed.

An application for plant breeders' rights for ‘DOW10’ has been lodged with the Australian Plant Breeders Rights Office, and was accepted on 24 Oct. 2005 (under Application No. 2005/288).

Parentage: The cultivar ‘DOW10’ was discovered in 2003 in Tuckombil, New South Wales, Australia, during a seedling selection of cultivated Tristaniopsis laurina (unpatented). The parent form is characterized by a dark brown bark color on stems prior to shedding, a medium green leaf color, a narrow leaf width with an oblanceolate to lanceolate leaf shape. Selection criteria for ‘DOW10’ were light brown bark on mature stems and large, glossy, dark green leaves combined with a vigorous growth rate.

Asexual reproduction. The new variety ‘DOW10’ was first asexually propagated by vegetative cuttings in the state of New South Wales, Australia in 2003. The distinctive characteristics of cultivar ‘DOW10’ have remained stable and true to type through successive cycles of asexual propagation.

### SUMMARY OF THE INVENTION

‘DOW10’ is a distinctive variety of Tristaniopsis laurina, which is characterized by its combination of light brown bark on mature stems shedding to reveal light gray brown colored immature bark and large, glossy, dark green leaves combined with a vigorous growth rate.

### BRIEF DESCRIPTION OF THE DRAWINGS

FIG. 1 shows a Tristaniopsis laurina ‘DOW10’ plant at approximately 18-months of age. The plant was propagated in a greenhouse and was transferred to the field at six months and grown out in full sun.

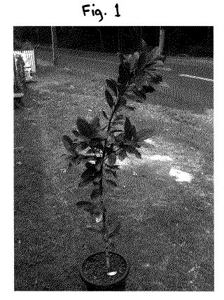

FIG. 2 shows the colors of new growth and older growth stems of ‘DOW10’0 after shedding of old bark.

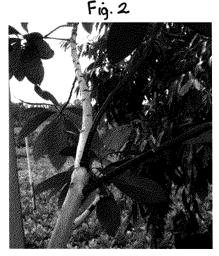

FIG. 3 shows the colors and patterns of shedding bark on stems of ‘DOW10’.

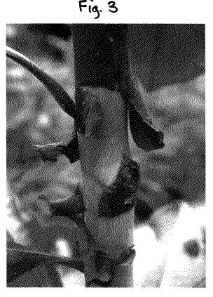

FIG. 4 shows the upper and lower side colors and shapes of leaves of ‘DOW10’.

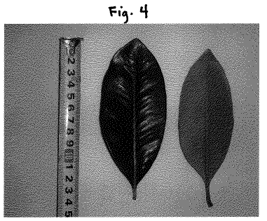

FIG. 5 shows the color of immature leaves of ‘DOW10’.

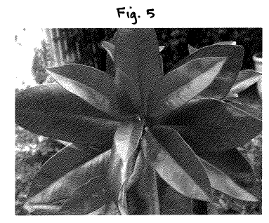

### DETAILED BOTANICAL DESCRIPTION OF THE VARIETY

The following is a detailed botanical description of a new and distinct variety of Tristaniopsis laurina known as ‘DOW10’ based upon observations of 18-month old plants grown in nursery pots in full sun in open beds in Theresa Park, New South Wales, Australia during 2005-2006 (as described below in connection with Table 1). Plant observations and descriptions were taken in winter 2006.

Those skilled in the art will appreciate that certain characteristics will vary with older or, conversely, with younger plants. ‘DOW10’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 1995 edition.

‘DOW10’ is an evergreen perennial Tristaniopsis laurina. ‘DOW10’ is an upright to spreading small tree with a vigorous growth rate. ‘DOW10’ has light brown bark which sheds in small strips to reveal a light gray brown colored immature layer of bark underneath. ‘DOW10’ has broad leaf blade widths with a dark green upperside leaf color and a contrasting lighter green leaf lower side color. ‘DOW10’ has an elliptic leaf shape with an acute apex shape. The surface of the leaf upperside of ‘DOW10’ has a glossy appearance and an absence of pubescence. These characteristics are unusual for Tristaniopsis laurina, as these plants usually have a darker bark color, narrower, lanceolate to oblanceolate shaped leaves and a less glossy, lighter green color leaf upperside than “DOW10’. A botanical description of ‘DOW10’ and comparisons with the parent form of Tristaniopsis laurina are provided below.

Technical Description of the Variety.

Plant characteristics: Growth habit upright to spreading, height medium (not yet recorded for a fully mature tree), typical of the species.

Stems: new stem has a smooth, glossy surface texture, color of young stem grayed purple (darker than RHS 187A), gradually changing with age to grayed orange (ca RHS 166A) and then further darkening as it is shed to brown (RHS 200A).The shedding of old bark reveals a light colored new bark layer which is lighter than gray brown (RHS 199D). Faint patterns remain on the new bark layer which correspond to the previously shed bark.

Leaves: Attitude horizontal to semi-erect, length medium (range 11 to 15cm), width broad (range 5.5 to 6.5cm), immature leaf upper side color grayed orange (ca RHS 166B to 175A), immature leaf lower side color yellow green (RHS 146D), mature leaf upper side color yellow green (RHS 147A) with strong surface glossiness, mature leaf lower side color yellow green (RHS 146C), variegation absent, shape elliptic, apex acute, cross section flat to slightly recurved, margin entire, very weak margin undulation, weak margin anthocyanin coloration on newly expanded leaf in winter, color grayed purple (RHS 187B), upper side midrib color yellow green (RHS 153D), lower side midrib color yellow green (RHS 152B), venation pinnate and corresponding to leaf color, petiole length short-medium (range 8-10mm).

Flowers and fruiting: Flowering characteristics of ‘DOW10’ have yet to be recorded.

Cold and Heat Tolerance: ‘DOW10’ has not yet been observed under all conditions.

‘DOW10’ is heat tolerant. It adapted well to the high heat conditions greater than 40 degrees Celsius during summer in Theresa Park, New South Wales, Australia without any noticeable damage. It has survived frosts to minus 4 degrees Celcius. Testing for environmental tolerances is ongoing.

Drought tolerance: Good drought tolerance typical of the species once established. Prefers moist conditions as it is naturally adapted to growing on river banks. ‘DOW10’ has not yet been observed under all conditions. Testing for environmental tolerances is ongoing.

Pest Resistance: No known pests.

Cultural conditions: ‘DOW10’ can tolerate low nutrient conditions; it does not like continually wet soil conditions, but can tolerate well-draining sandy soils to very heavy clay soils.

Comparisons with Other Tristaniopsis.

‘DOW10’ is a more attractive ornamental tree as compared with the parent Tristaniopsis laurina (unpatented, wild type). ‘DOW10’ is characterized by its combination of light brown bark on mature stems shedding to reveal light gray brown colored immature bark and large, glossy, dark green leaves combined with a vigorous growth rate as compared with Tristaniopsis laurina. The appearance of ‘DOW10’ is lush with broad glossy, dark green leaves, whereas Tristaniopsis laurina has narrower, less glossy, lighter green leaves (see Table 1).

Tristaniopsis laurina (unpatented, wild type) is the most similar comparator variety to ‘DOW10’. In comparison with Tristaniopsis laurina, cultivar ‘DOW10’ has narrower, less glossy, lighter green leaves (see Table 1).

A comparative trial of Tristaniopsis cultivar ‘DOW10’ with Tristaniopsis laurina (unpatented, wild type) is currently being carried out in Theresa Park, New South Wales, Australia. The initial data are presented in Table 1 below.

TABLE 1

Tristaniopsis Variety

| Characteristic | ‘DOW10’ | Tristaniopsis laurina |
|---|---|---|
| Stem: color of bark | light brown | dark brown |
| Leaf: width | broad | narrow-medium |
| Leaf: color | dark green | medium green |
| Leaf: glossiness | strong | medium |
| Leaf: shape | elliptic | lanceolate-oblanceolate |

That which is claimed is:

1. A new and distinct variety of Tristaniopsis laurina plant named ‘DOW10’, substantially as described and illustrated herein.
